SEQUENCE LISTING (24 sequences)

SEQ ID NO: 1 - DNA, 1516 bp, homo sapiens
  misc_feature: mRNA LYZ human
aaatactggggccagctcaccctggtcagcctagcactctgacctagcagtcaacatgaaggctctcattgttctggggcttgtcctcctttctgttacggtccagggcaaggtctttgaaaggtgtgagttggccagaactctgaaaagattgggaatggatggctacaggggaatcagcctagcaaactggatgtgtttggccaaatgggagagtggttacaacacacgagctacaaactacaatgctggagacagaagcactgattatgggatatttcagatcaatagccgctactggtgtaatgatggcaaaaccccaggagcagttaatgcctgtcatttatcctgcagtgctttgctgcaagataacatcgctgatgctgtagcttgtgcaaagagggttgtccgtgatccacaaggcattagagcatgggtggcatggagaaatcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttctttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatacgattatgcccaatattaagtaaaaaatataagaaaaggttatcttaaatagatcttaggcaaaataccagctgatgaaggcatctgatgccttcatctgttcagtcatctccaaaaacagtaaaaataaccactttttgttgggcaatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaattaaaggctttgcaacac

SEQ ID NO: 2 - DNA, 2395 bp, homo sapiens
  misc_feature: G6PD mRNA human isoform A
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcc

SEQ ID NO: 3 - DNA, 2270 bp, homo sapiens
  misc_feature: G6PD mRNA human isoform B
cgagaactcgggaagccggcgagaagtgtgaggccgcggtagggccgcatcccgctccggagagaagtctgagtccgccaggctctgcaggcccgcggaagctcgacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcc

SEQ ID NO: 4 - DNA, 1937 bp, homo sapiens
  misc_feature: mRNA 6PGD
ggccgcagtttctggagggagccgctgcgggtctttccctcactcgtcctccgcgcgtcgccgctcttcggttctgctctgtccgccgccatggcccaagctgacatcgcgctgatcggattggccgtcatgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggtggcaccgtgtcatcctcgtcatacaatgcctgatcatgctgctcctgtcaccctccacgattccacagaccaggacattccatgtgcctcatggcactgccacctggccctttgccctattttctgttcagttttttaaaagtgttgtaagagactcctgaggaagacacacagtttatttgtaaagtagctctgtgagagccaccatgccctctgcccttgcctcttgggactgaccaggagctgctcatgtgcgtgagagtgggaaccatctccttgcggcagtggcttccgcgtgccccgtgtgctggtgcggttcccatcacgcagacaggaagggtgtttgcgcactctgatcaactggaacctctgtatcatgcggctgaattccctttttcctttactcaataaaagctacatcacactga

SEQ ID NO: 5 - DNA, 2971 bp, homo sapiens
  misc_feature: mRNA LILRB1 human, isoform 1
gaggaggaacagaaaagaaaagaaaagaaaaagtgggaaacaaataatctaagaatgaggagaaagcaagaagagtgacccccttgtgggcactccattggttttatggcgcctctactttctggagtttgtgtaaaacaaaaatattatggtctttgtgcacatttacatcaagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggccccaggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcactctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaatcctccagtgtgactcacaggtggcatttgatggcttcagtctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaa

SEQ ID NO: 6 - DNA, 2802 bp, homo sapiens
  misc_feature: mRNA LILRB1 human, isoform 2
ctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggccccaggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcactctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaatcctccagtgtgactcacaggtggcatttgatggcttcagtctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctgcaggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaa

SEQ ID NO: 7 - DNA, 2859 bp, homo sapiens
  misc_feature: mRNA LILRB1 human, isoform 3
aagtcaacttttcttccctatttccctgcatttctcttctgtgctcgctgccacacgcagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggccccaggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcactctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaatcctccagtgtgactcacaggtggcatttgatggcttcagtctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctgcaggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaa

SEQ ID NO: 8 - DNA, 2859 bp, homo sapiens
  misc_feature: mRNA LILRB1 human, isoform 4
aagtcaacttttcttccctatttccctgcatttctcttctgtgctcgctgccacacgcagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggccccaggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcactctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaatcctccagtgtgactcacaggtggcatttgatggcttcagtctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaa

SEQ ID NO: 9 - DNA, 2179 bp, homo sapiens
  misc_feature: mRNA of TKT, variant 1
gatccgagccccgcctcctccccctgccccgcctctcccatccccgccccgccccgcccggcgacttaacgcgcccccgccccgcgcccggcctcggcagccgcctgtcgccgcgggagcagccgctatctctgtgtgtccgcgtgtgcgcccggtccccgcctgccgcaccatggagagctaccacaagcctgaccagcagaagctgcaggccttgaaggacacggccaaccgcctacgtatcagctccatccaggccaccactgcggcgggctctggccaccccacgtcatgctgcagcgccgcagagatcatggctgtcctctttttccacaccatgcgctacaagtcccaggacccccggaatccgcacaatgaccgctttgtgctctccaagggccatgcagctcccatcctctacgcggtctgggctgaagctggtttcctggccgaggcggagctgctgaacctgaggaagatcagctccgacttggacgggcacccggtcccgaaacaagctttcaccgacgtggccactggctccctgggccagggcctcggggccgcttgtgggatggcctacaccggcaaatacttcgacaaggccagctaccgagtctattgcttgctgggagacggggagctgtcagagggctctgtatgggaggccatggccttcgccagcatctataagctggacaaccttgtggccattctagacatcaatcgcctgggccagagtgacccggccccactgcagcaccagatggacatctaccagaagcggtgcgaggccttcggttggcatgccatcatcgtggatggacacagcgtggaggagctgtgcaaggcctttggccaggccaagcaccagccaacagccatcattgccaagaccttcaagggccgagggatcacgggggtagaagataaggagtcttggcatgggaagcccctccccaaaaacatggctgagcagatcatccaggagatctacagccagatccagagcaaaaagaagatcctggcaacccctccacaggaggacgcaccctcagtggacattgccaacatccgcatgcccagcctgcccagctacaaagttggggacaagatagccacccgcaaggcctacgggcaggcactggccaagctgggccatgccagtgaccgcatcatcgccctggatggggacaccaaaaattccaccttctcggagatcttcaaaaaggagcacccggaccgcttcatcgagtgctacattgctgagcagaacatggtgagcatcgcggtgggctgtgccacccgcaacaggacggtgcccttctgcagcacttttgcagccttcttcacgcgggcctttgaccagattcgcatggccgccatctccgagagcaacatcaacctctgcggctcccactgcggcgtttccatcggggaagacgggccctcccagatggccctagaagatctggctatgtttcggtcagtccccacatcaactgtcttttacccaagtgatggcgttgctacagagaaggcagtggaactagccgccaatacaaagggtatctgcttcatccggaccagccgcccagaaaatgccatcatctataacaacaatgaggacttccaggtcggacaagccaaggtggtcctgaagagcaaggatgaccaggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctggcagttaaccgggtaccaagaagtgggaagccggctgagctgctgaagatgtttggtatcgacagggatgccattgcacaagctgtgaggggcctcatcaccaaggcctagggcgggtatgaagtgtggggcgggggtctatacattcctgagattctgggaaaggtgctcaaagatgtactgagaggaggggtaaatatatgttttgagaaaaatgaattggccctgaaaaaaaaaaaaaaaaaa

SEQ ID NO: 10 - DNA, 2957 bp, homo sapiens
  misc_feature: Variant 2 de TKT
gatccgagccccgcctcctccccctgccccgcctctcccatccccgccccgccccgcccggcgacttaacgcgcccccgccccgcgcccggcctcggcagccgcctgtcgccgcgggagcagccgctatctctgtgtgtccgcgtgtgcgcccggtccccgcctgccgcaccatggagagctaccacaagcctgaccagcagaagctgcaggccttgaaggacacggccaaccgcctacgtatcagctccatccaggccaccactgcggcgggctctggccaccccacgtcatgctgcagcgccgcagagatcatggctgtcctctttttccacaccatgcgctacaagtcccaggacccccggaatccgcacaatgaccgctttgtgctctccaagggccatgcagctcccatcctctacgcggtctgggctgaagctggtttcctggccgaggcggagctgctgaacctgaggaagatcagctccgacttggacgggcacccggtcccgaaacaagctttcaccgacgtggccactggctccctgggccagggcctcggggccgcttgtgggatggcctacaccggcaaatacttcgacaaggccagctaccgagtctattgcttgctgggagacggggagctgtcagagggctctgtatgggaggccatggccttcgccagcatctataagctggacaaccttgtggccattctagacatcaatcgcctgggccagagtgacccggccccactgcagcaccagatggacatctaccagaagcggtgcgaggccttcggttggcatgccatcatcgtggatggacacagcgtggaggagctgtgcaaggcctttggccaggccaagcaccagccaacagccatcattgccaagaccttcaagggccgagggatcacgggggtagaagataaggagtcttggcatgggaagcccctccccaaaaacatggctgagcagatcatccaggagatctacagccagatccagagcaaaaagaagatcctggcaacccctccacaggaggacgcaccctcagtggacattgccaacatccgcatgcccagcctgcccagctacaaagttggggacaagatagccacccgcaaggcctacgggcaggcactggccaagctgggccatgccagtgaccgcatcatcgccctggatggggacaccaaaaattccaccttctcggagatcttcaaaaaggagcacccggaccgcttcatcgagtgctacattgctgagcagaacatggtgagcatcgcggtgggctgtgccacccgcaacaggacggtgcccttctgcagcacttttgcagccttcttcacgcgggcctttgaccagattcgcatggccgccatctccgagagcaacatcaacctctgcggctcccactgcggcgtttccatcggggaagacgggccctcccagatggccctagaagatctggctatgtttcggtcagtccccacatcaactgtcttttacccaagtgatggcgttgctacagagaaggcagtggaactagccgccaatacaaagggtatctgcttcatccggaccagccgcccagaaaatgccatcatctataacaacaatgaggacttccaggtcggacaagccaaggtggtcctgaagagcaaggatgaccaggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctggcagttaaccgggtaccaagaagtgggaagccggctgagctgctgaagatgtttggtatcgacagggatgccattgcacaagctgtgaggggcctcatcaccaaggcctagggcggccagtgcaggttctggggaagaagcgtggagggtgcgctagggaggctggtgtccttggtcctgggccaggacctgggtgtcccaagtccccttggaatcacctgcaactgccctcaacctccagaactatcgctgcctcccattcatcaccaggtgacatttgactgcaacctgccttctagctgagaggctgagacctacatccctcattgtgacctcagtccacctggccctgagcgggctggggaactgcctcagtctggaagctgaccaggcactctcagggccgccccacctcccccaagtccccacagccttgcagtcaggtctcctgggatagggaggttcacttgcttgttgtccctcgtccttgtcatatccttttaactaggcatctcagagaagcagagacagggcagccttcgtcctgggggaaaagggaccctcaggatggcatgagaggtcctcaatcccaagtgtggaactgtccccctcaacttgttaaaatgcagatttctgggtcttgccaatggggcctgggactccatgtgacaactggcccaggagcttctgatgtcacacagaattctgcagtcccaagctccagccccgacctgctctgctgttcctaggtgactgccctcacactgctgaccacagtggatttctccccctgctgctcgggctcagctggggtcagccctgcttataaggtcaactgtgcaaaaccttatactggccaagaacaaactagtgctgggggaggagggctgggtgccccggccactggtggagtccccaggaaatcctcagagctgttgcgaggatgagacacatttgtggacacgtccacctgtcctcctgaccgtctggagagaataaacctgtcaaagccaaggtcaaaaaa

SEQ ID NO: 11 - DNA, 1205 bp, homo sapiens
  misc_feature: mRNA of CEACAM4
acagaaggaggaaggtcagcagccccgacagccgacagtcacagcagctctgacaagagcgttcctggagcccagctcctctccacagaggacaagcaggcagcagagaccatgggccccccctcagccgctccccgtggagggcacaggccctggcaggggctcctgatcacagcctcacttttaaccttctggcacccgcccaccactgtccagttcactattgaagccctgccgtccagtgctgcagagggaaaggatgttcttctactggcctgcaatatttcagaaactattcaagcctattattggcacaaggggaaaacggcagaagggagccctctcattgctggttatataacagacattcaagcaaatatcccaggggccgcatacagtggtcgagagacagtataccccaatggatccctgctgttccaaaacatcaccctggaggacgcaggatcctacaccctacgaaccataaatgccagttacgactctgaccaagcaactggccagctccacgtacaccaaaacaacgtcccaggccttcctgtgggggccgtcgctggcatcgtgactggggtcctggttggggtggctctggtggccgccctggtgtgttttctgcttctctccaggactggaagggccagcatccagcgtgacctcagggagcagccgcccccagcctccacccctggccatggtccctctcacagatccaccttctcggcccctctacccagccccagaacagccactcccatctatgaggaattgctatactctgatgcaaacatttactgccagatcgaccacaaagcagatgtggtctcttaggttcctctgggagctgctcttgtgggttgatggagcgtccccaaagctcccagccctggggacggggaaggacatggagcctgagccagagaaccagctctgagtcctgaggagacacaggcctggggacagggagggatgggagtccctgctgaatatctggagaccctgacaggttgccctgggctctgggtgggccgggacaaaggcctctcatcaccacaggaagcgggggcttgcaaggaaagtgaatgggcctgtggcccacccggggtcaccaggaaaggatctgaataaagaggacccttcctctcattggctctttttctgctcacgggaacttagcagaaactcacctg

SEQ ID NO: 12 - protein, 148 aa, homo sapiens
  misc_feature: protein Lysozyme
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 13 - protein, 545 aa, homo sapiens
  misc_feature: isoform A, protein G6PD
MGRRGSAPGNGRTLRGCERGGRRRRSADSVMAEQVALSRTQVCGILREELFQGDAFHQSDTHIFIIMGASGDLAKKKIYPTIWWLFRDGLLPENTFIVGYARSRLTVADIRKQSEPFFKATPEEKLKLEDFFARNSYVAGQYDDAASYQRLNSHMNALHLGSQANRLFYLALPPTVYEAVTKNIHESCMSQIGWNRIIVEKPFGRDLQSSDRLSNHISSLFREDQIYRIDHYLGKEMVQNLMVLRFANRIFGPIWNRDNIACVILTFKEPFGTEGRGGYFDEFGIIRDVMQNHLLQMLCLVAMEKPASTNSDDVRDEKVKVLKCISEVQANNVVLGQYVGNPDGEGEATKGYLDDPTVPRGSTTATFAAVVLYVENERWDGVPFILRCGKALNERKAEVRLQFHDVAGDIFHQQCKRNELVIRVQPNEAVYTKMMTKKPGMFFNPEESELDLTYGNRYKNVKLPDAYERLILDVFCGSQMHFVRSDELREAWRIFTPLLHQIELEKPKPIPYIYGSRGPTEADELMKRVGFQYEGTYKWVNPHKL

SEQ ID NO: 14 - protein, 515 aa, homo sapiens [sequence omitted]

SEQ ID NO: 15 - protein, 483 aa, homo sapiens
  misc_feature: Protein 6PGD
MAQADIALIGLAVMGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHGGTVSSSSYNA

SEQ ID NO: 16 - protein, 650 aa, homo sapiens
  misc_feature: Protein LILRB1, isoform 1
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 17 - protein, 652 aa, homo sapiens
  misc_feature: Protein LILRB1, isoform 2
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 651 aa, homo sapiens
  misc_feature: Protein LILRB1, isoform 3
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 19 - protein, 651 aa, homo sapiens
  misc_feature: Protein LILRB1, isoform 4
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 20 - protein, 623 aa, homo sapiens
  misc_feature: Variant 1 de TKT
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAEQNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 21 - protein, 623 aa, homo sapiens; identical to SEQ ID NO: 20
  misc_feature: Variant 2 de TKT

SEQ ID NO: 22 - protein, 244 aa, homo sapiens
  misc_feature: CEACAM4
MGPPSAAPRGGHRPWQGLLITASLLTFWHPPTTVQFTIEALPSSAAEGKDVLLLACNISETIQAYYWHKGKTAEGSPLIAGYITDIQANIPGAAYSGRETVYPNGSLLFQNITLEDAGSYTLRTINASYDSDQATGQLHVHQNNVPGLPVGAVAGIVTGVLVGVALVAALVCFLLLSRTGRASIQRDLREQPPPASTPGHGPSHRSTFSAPLPSPRTATPIYEELLYSDANIYCQIDHKADVVS

SEQ ID NO: 23 - DNA, 938 bp, homo sapiens
  misc_feature: ELANE
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaaaaaaaaaaaaaaaa

SEQ ID NO: 24 - protein, 267 aa, homo sapiens
  misc_feature: ELANE
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

(19) United States
(12) Patent Application Publication
(10) Pub. No.: US 2016/0017429 A1
(43) Pub. Date: Jan. 21, 2016

(54) METHOD FOR PROGNOSING THE SURVIVAL OF PATIENTS SUFFERING FROM CHRONIC MYELOMONOCYTIC LEUKAEMIA

(71) Applicants: Centre Hospitalier Universitaire Nîmes, Nîmes (FR); ACOBIOM, Grabels (FR)

(72) Inventors: Davidè PIQUEMAL, Saint-Christol-lès-Alès (FR); Eric JOURDAN, Nîmes (FR); Thérèse COMMES, Vailhauquès (FR); Elias BOU SAMRA, Broumana (LB)

(73) Assignees: Centre Hospitalier Universitaire Nîmes, Nîmes (FR); ACOBIOM, Grabels (FR)

(21) Appl. No.: 14/378,727

(22) PCT Filed: Feb. 18, 2013
(86) PCT No.: PCT/EP2013/053204

§ 371 Date: Aug. 14, 2014

(30) Foreign Application Priority Data
Feb. 17, 2012 (EP) 12305187.2

(51) Int. Cl. C12Q 1/68; G01N 33/574

(52) U.S. Cl. CPC C12Q 1/6886; G01N 33/57426; C12Q 2600/158; C12Q 2600/106; G01N 2800/52; G01N 2800/60

(57) ABSTRACT

The present invention relates to a method of prognostic of the survival of human subject suffering from chronic myelomonocytic leukemia (CMML) based on the differential expression of six genes in a test sample of PBMC cells obtained from said human subject and in a control sample of normal cells, wherein said expression level indicates if the human subject from which the test sample has been obtained will have long-term or short-term survival.

### TECHNICAL BACKGROUND OF THE INVENTION

Chronic myelomonocytic leukemia (CMML) is a clonal hematopoietic stem cell disorder frequently seen in the elderly people. First considered as a myelodysplasic disorder in the French. American british (FAB), CMML was reclassified by the World Health Organization (WHO) as myelodysplasic/myeloproliferative entity. This reclassification allows considering the heterogeneity of the CMML syndrome in diagnosis and prognosis. Despite this heterogeneity, the diagnosis of CMML is definitely straightforward in the presence of a combination of persistent blood monocytosis and fewer than 20% blasts in peripheral blood and bone marrow. According to WHO criteria, blasts include myeloblasts, monoblasts and promonocytes. The myeloid compartment is frequently associated with cytogenetic abnormality that helps to confirm the CMML diagnosis. Thus, CMML is mainly characterized by a persistent peripheral monocytosis (>1×10⁹/l), less than 20% blasts in blood and bone marrow, and a variable degree of dysplasia in one or more myeloid lineages. However CMML patients often showed heterogeneity in cytogenetics, and these cytogenetic markers have therefore a poor prognostic value. Major difficulties are faced in the clinical classification of this disease and the variable risk of its progression.

Molecular studies based on mutation identification may provide promising insights in the diagnosis and prognosis process. Twenty two percent of patients exhibit point mutations of RAS genes (NRAS, KRA) at diagnosis or during the disease course and as many as 50% present TET2 mutations (Ricci, C. et al., Clincal Cancer Research, 2010). More recently, by applying next-generation sequencing (NGS) technology to characterize molecular mutations, Kohlmann et al. detected at least one aberration in 72.8% of CMML cases, including in the ten-eleven translocation 2 (TET2) gene. According to them, patients carrying these mutations present a better outcome contrary to Kosmider et al. where TET2 mutations are linked to a poor prognosis (Kohlmann, A. et al., Journal of Clinical Oncology, 2010; Tefferi A et al., Leukemia, 2009; Kosmider O et al., Haematologica, 2009). In absence of major prognostic markers, the physicians face major problems in evaluating the variable risk of progression of this disease to acute myeloid leukemia (AML): the disease is greatly heterogeneous in term of clinical course, a part of the patients displaying an indolent and stable disease, other displaying a more aggressive disease. Criteria for initiating a therapy in CMML are not well established, and depend on the physician's experience.

There is therefore a need of a rapid and reliable prognostic method enabling to predict the survival chances of a patient suffering from CMML and/or the suitability of said patient for a drug trial.

Analysis of gene expression profiles (GEP) is very promising in the medical field. It helps the discovery of new tools for applied therapy, notably new prognostic and diagnostic markers, and highlights evaluation criteria for treatments and disease follow-up. However, despite the highly documented data concerning acute leukemia, slight information till now is known about myelodysplastic syndromes, and particularly about CMML.

As shown in the herein presented results, the present inventors have found 5 new strong molecular prognostic markers including the G6PD, 6PGD, TKT, CEACAM4 and ELANE genes. All are predominantly linked to a promyelocytic phenotype according to Amazonia, the public DNA microarray database. Likewise, a clear distinction between two sets of patients has been observed for the first time, depending reliably on the expression levels of each of these markers: patients having a “bad” prognosis of survival, with median time survival (MTS) of 21 months (less than two years), and patients having a “good” prognosis of survival, with MTS of 83 months (almost 4 years).

This represents an important and medically useful discovery as it will enable to determine prior to the treatment which patients will fail therapeutic treatment, thus saving them from up to a year of an expensive treatment with significant side effects.

### FIGURE LEGENDS

FIG. 1. Supervised clustering of CMML samples using 28 significantly expressed genes (FDR<5%). Red and green indicate over and under-expressed genes, respectively. Each row represents a single gene probe (28 genes) and each column represents a distinct CMML sample (32 samples). Samples were clustered into 2 subtypes, A and B. Subtypes A and B group 13 and 19 CMML patients, respectively.

FIG. 2. Kaplan-Meïer estimates of overall survival (OS). The index computation based on the expression data of the 5 selected genes (TKT, G6PD, ELANE, PGD and CEACAM4) allowed the discrimination between two distinct groups of patients. Patients were equally distributed (N=16 in each group). We characterize a good survival group (dotted grey) with a low index score and 94% probability of survival ( 15/16), and a poor survival group (black) with a high index score and 19% probability of survival ( 3/16). A P-value of 0.007 was obtained.

FIG. 3. Microarray expression of selected genes. Expression histograms of five specific genes TKT, G6PD, PGD, ELANE and CEACAM4 in various normal haematological tissues. Histograms were obtained from the Amazonia website from the HG-U133 Plus 2.0; Affymetrix (Santa Clara, Calif.) oligonucleotide microarray datasets.

FIG. 4. Kaplan-Meïer estimates of overall survival (OS). A) The index computation based on the expression data of the 5 selected genes (TKT, G6PD, ELANE, PGD and CEACAM4) in the new cohort of 21 CMML samples allowed the discrimination between two distinct groups of patients. We characterized a good survival group (green) with a low index score and 56% probability of survival ( 5/9), and a poor survival group (red) with a high index score and 25% probability of survival ( 3/12). A P-value of 0.03 was obtained. B) The index computation based on the expression data of the 5 selected genes (TKT, G6PD, ELANE, PGD and CEACAM4) in both mixed cohorts of 53 CMML samples allowed the discrimination between two distinct groups of patients. We characterized a good survival group (green) with a low index score and 80% probability of survival ( 20/25), and a poor survival group (red) with a high index score and 21% probability of survival ( 6/28). A P-value of 0.002 was obtained.

### DESCRIPTION OF THE INVENTION

Interestingly, the present inventors have found that the survival chances of patients suffering from chronic myelomonocytic leukaemia can be assessed on the simple analysis of the expression level in PBMC cells of a set of 5 genes or homologous thereof, and comparison with the expression level of the same genes in PBMCs of healthy patients.

In a first aspect, the present invention thus relates to a method for in vitro determining the prognosis of chronic myelomonocytic leukaemia (CMML) in a human patient suffering thereof, comprising at least the following steps:

a) measuring in a test sample of said patient the expression levels of at least two genes chosen in the group consisting of: G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof,
b) comparing said expression levels to the expression level of said at least two genes in at least one control sample obtained from at least one known healthy human subject,
c) predicting the outcome of the chronic myelomonocytic leukaemia in said patient and/or the suitability of said patient for a drug trial.

More precisely, the present invention relates to a method for in vitro determining the prognosis of CMML in a human patient suffering thereof, comprising at least the following steps:

a) obtaining a test sample from said human patient,
b) measuring the expression profile comprising at least two genes chosen in the group consisting of: G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof in said test sample,
c) comparing said expression profile with at least one reference profile,
c) predicting the outcome of CMML in said patient.

According to the invention, a “CMML suffering patient” is a human subject showing persistent blood monocytosis and fewer than 20% blasts (myeloblasts, monoblasts and/or promonocytes) in peripheral blood and bone marrow.

The present invention enables to “prognose” (or to “determine the prognosis” of) the future life-span of a patient suffering from CMML, i.e. to predict the outcome of said disease in terms of month-survival for said patient, said patient being treated or not against this disease.

As used in the present application, the term “test sample” designates any sample that may be taken from a CMML suffering patient, such as a serum sample, a plasma sample, a urine sample, a blood sample, a lymph sample, or a biopsy. Preferred test sample for the determination of the gene expression levels is blood sample, more preferably a peripheral blood sample comprising peripheral blood mononuclear cells (PBMC) or whole blood. Such PBMC samples can be obtained by a completely non-invasive harmless blood collection from the patient, followed by a classical ficoll separation as described in Cytotherapy (Janssen W E et al., 2010). More preferably, purity of the PBMC sample is up to 70%, preferably up to 80% and more preferably up to 90% as classically obtained by Ficoll purification processes.

As used herein, the term “expression profile” designates the expression levels of a group of at least two genes chosen in the group consisting of G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof.

In a preferred embodiment, the expression level of at least three, preferably four, and more preferably five of the genes chosen in the group consisting of G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof is measured in the method of the invention. In a more preferred embodiment, the expression level of the five genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) is measured, and the expression profile of the invention therefore consists of the expression level of these five genes.

A sixth gene can also be used in the method of the invention. This sixth gene is the LYZ gene of SEQ ID NO:1. Thus, in a more preferred embodiment, the expression level of at least four, preferably five, and more preferably six of the genes chosen in the group consisting of LYZ (SEQ ID NO:1), G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof is measured in the method of the invention. In a more preferred embodiment, the expression level of the six genes LYZ (SEQ ID NO:1, G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) is measured, and the expression profile of the invention therefore consists of the expression level of these five genes.

The 6 genes that were determined by the Inventors to be able to discriminate between patients having a bad or a long survival are listed in the following table 1:

TABLE 1

| Symbole | Accession number NCBI | Encoded protein |
|---|---|---|
| LYZ | NM_000239.2 (SEQ ID NO: 1) | Lysozyme C (enzyme EC 3.2.1.17) |
| | | Muramidase |
| | | N-acetylmuramide glycanhydrolase |
| | | NP_000230.1 (SEQ ID NO: 12) |
| G6PD | NM_000402.3 isoform A (SEQ | Glucose-6-phosphate dehydrogenase (enzyme EC |
| | ID NO: 2) | 1.1.1.49) |
| | NM_001042351.1 isoform B (SEQ | Isoform A: NP_000393.4 (SEQ ID NO: 13) |
| | ID NO: 3) | Isoform B: NP_001035810.1 (SEQ ID NO: 14) |
| 6PGD (or | NM_002631.2 (SEQ ID NO: 4) | Phosphogluconate dehydrogenase (PGDH) |
| PGD) | | 6 phosphogluconate dehydrogenase decarboxylating |
| | | (6PGD) |
| | | EC 1.1.1.44 |
| | | NP_002622.2 (SEQ ID NO: 15) |
| ELANE | NM_001972.2 (SEQ ID NO: 23) | neutrophil-expressed Elastase |
| | | NP_001963.1(SEQ ID NO: 24) |
| TKT | NM_001064.3 (SEQ ID NO: 9) | Transketolase humaine |
| | NM_001135055.2 (SEQ ID NO: 10) | NP_001055.1: variant 1 (SEQ ID NO: 20) |
| | | NP_001128527.1: variant (SEQ ID NO: 21) |
| CEACAM4 | NM_001817.2 (SEQ ID NO: 11) | Homo sapiens carcinoembryonic antigen-related cell |
| | | adhesion molecule 4 |
| | | NP_001808.2 (SEQ ID NO: 22) |

The term “homologous” refers to sequences that have sequence similarity. The term “sequence similarity”, in all its grammatical forms, refers to the degree of identity or correspondence between nucleic acid sequences. In the context of the invention, two nucleic acid sequences are “homologous” when at least about 80%, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, alternatively at least about 99% of the nucleic acids are similar. Preferably the similar or homologous nucleic acid sequences are identified by alignment using, for example, the algorithm of Needleman-Wurisch.

The expression levels (or expression profile) may be determined by any technology known by a man skilled in the art. In particular, each gene expression level may be measured at the genomic and/or nucleic and/or proteic level.

In a preferred embodiment, measuring the expression levels of the said genes (or the expression profile) is performed by measuring the amount of nucleic acid transcripts of each gene. The amount of nucleic acid transcripts of each gene can be measured by any technology known by a man skilled in the art. In particular, the measure can be carried out directly on extracted messenger RNA (mRNA) sample, or on retrotranscribed complementary DNA (cDNA) prepared from extracted mRNA by technologies well-known in the art. From the mRNA or cDNA sample, the amount of nucleic acid transcript may be measured using any technology known by a man skilled in the art, including microarrays, quantitative PCR, DNA chips, hybridization wit labelled probes, or flow lateral dipstick (Surasilp T. et al., Mol Cell Probes. 2011). In a preferred embodiment, the expression levels are determined using quantitative PCR. Quantitative or real-time, PCR is a well-known and easily available technology for those skilled in the art and does therefore not need a precise description.

In this case, the measuring level is preferably performed by including an invariant endogenous reference gene (such as the RPS19 gene), in the RNA/DNA detection assay to correct for sample to sample variations in PCR (or hybridization) efficiency and errors in sample quantification.

In another preferred embodiment, the expression levels of the said genes are determined by the use of nucleic microarrays.

According to the invention, a “nucleic microarray” consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”), and the oligonucleotides may be about 25 to about 60 base pairs or less in length.

To determine the expression levels of define gene in a target nucleic sample, said sample can be labelled, contacted with the microarray in hybridization conditions, leading to the formation of complexes between target nucleic acids that are complementary to probe sequences attached to the microarray surface. The presence of labelled hybridized complexes can then be detected. Many variants of the microarray hybridization technology are available to the man skilled in the art.

In a preferred embodiment, the nucleic acid microarray is an oligonucleotide microarray comprising or consisting of 5 oligonucleotides specific for the 5 genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) (see Table 1 below). Preferably, the microarray also comprises the LYZ gene of SEQ ID NO:1.

Preferably, the oligonucleotides are about 50 bases in length. It is acknowledged that the nucleic acid microarray, or oligonucleotide microarray of the invention encompass the microarrays specific for the homologous genes as defined below.

Suitable oligonucleotides may be designed, based on the genomic sequence of each gene (see Genbank accession numbers), using any method of microarray oligonudeotide design known in the art. In particular, any available software developed for the design of microarray oligonucleotides may be used, such as, for instance, the OligoArray software (available at http://berry.engin.umich.edu/oligoarray/), the GoArrays software (available at http://www.isima.fr/bioinfo/goarrays/), the Array Designer software (available at http://www.premierbiosoft.com/dnamicroarray/index.html), the Primer3 software (available at http://frodo.wi.mit.edu/prirmer3/primer3_code.html), or the Promide software (available at http://oligos.molgen.mpg.de/).

In another preferred embodiment, measuring the expression levels of the said gene is performed by measuring the respective levels of the encoded proteins of the said genes, for example by employing antibody-based detection methods such as immunohistochemistry or western blot analysis, proteic microarray, flow cytometry or flow lateral dipstick (Surasilp T et al., Mol Cell Probes. 2011).

Said encoded proteins are namely: SEQ ID NO: 12 for the LYZ protein, SEQ ID NO:13 or 14 for the G6PD protein, SEQ ID NO:15 for the 6PGD protein, SEQ ID NO:24 for the ELANE protein, SEQ ID NO:20 or 21 for the TKT protein and SEQ ID NO:22 for the CEACAM4 protein.

For expression profiling experiments, antibodies, aptamers, or affibodies microarrays are mainly used, most of the time antibodies microarrays (Hall et al, 2007). The antibodies, aptamers, or affibodies are attached to various supports using various attachment methods, using a contact or non-contact spotter (Hall et al, 2007). Examples of suitable supports include glass and silicon microscope slides, nitrocellulose, microwells (for instance made of a silicon elastomer) (Hall et al, 2007). For glass and silicon microscope slides, a coating is generally added. Examples of coatings for random attachment (i.e. resulting in a random orientation of attached proteins to the support) include aldehyde- and epoxy-derivatized coatings for random attachment through amines, and nitrocellulose, gel pads or poly-L-lysine coatings (Hall et al, 2007). Examples of coatings for non random attachment (i.e. resulting in a uniform orientation of attached proteins to the support) include nickel coating fro use with His6-tag proteins, and streptavidin coating for use with biotinylated proteins (Hall et al, 2007). For detection, two main technologies can be used: 1) direct labelling, single capture assays and 2) dual-antibody sandwich immunoassays (Kingsmore, 2006). In direct labelling, single capture assays, proteins contains in one or more samples are labelled with distinct labels (generally fluorescent or radioisotope labels), hybridized to the microarray, and labelled hybridized proteins are directly detected (Kingsmore, 2006). In dual-antibody sandwich immunoassays, the sample is hybridized to the microarray, and a secondary tagged antibody is added. A third labelled (generally fluorescent or radioisotope label) antibody specific for the tag of the secondary antibody is then used for detection (Kingsmore, 2006). Further details concerning antibodies microarrays may be found in Haab, 2005 and Eckel-Passow et al, 2005. Examples of commercial antibody microarrays include those commercialized by Clontech Laboratories, Invitrogen, Eurogentec, Kinexus etc. . . . .

The determination of the survival prognostic according to the method of the invention is carried out thanks to the comparison of the expression profile of the above-mentioned genes with at least one reference profile.

A reference profile is, in the context of the present invention, obtained from a “control sample”, i.e. from a test sample obtained from a human subject who is known to be healthy. Preferably, said reference profile has been obtained from several healthy subjects (for example from at least 5 healthy subjects) by measuring the expression level of each gene and by calculating a mean thereof. As used herein, the terms “a control sample of a known healthy human subject” therefore mean “at least one control sample of at least one known healthy human subject”.

The comparison of a tested sample with a control sample (or of a tested expression profile to a reference expression profile) can be done using statistical models or machine learning methods which aim is to predict a clinical response (e.g.: 0 if bad prognosis, 1 if good prognosis) based on a combination of the explanatory variables (the genes). Statistical models such as logistic regression and fisher linear discriminant analysis are particularly relevant to predict outcome. Other discriminating algorithms include kNN (k nearest neighbour), decision trees, SVM (support vector machine), NN (neural networks) and forest. The PLS regression, MIPP, sparse linear discrimination and PAM (predictive analysis of microarrays) are particularly relevant to give prediction in the case of pangenomic analysis with small reference samples. To ensure that the predictor is robust, cross validation methods such as leave-one-out should be applied to the models.

The comparison step of the method of the invention can be for example performed by calculating the ratio between the expression level of each gene in the tested sample and in the control reference sample.

In a preferred embodiment, higher expression level of at least two, preferably three, more preferably four genes and even more preferably five genes chosen in the group consisting of: G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) or homologous thereof in said test sample, as compared to said control sample, indicates a long-term survival of said human patient.

As mentioned herein, the term “long-term survival” refers to survival of at least 70 months, preferably 75 months and more preferably 80 months after the sample collection has been performed, the patient being treated or not.

As used herein, the term “higher expression level” means that the expression level of a gene in said test sample is strictly superior to the one in said control sample; because said gene is up-regulated in said test sample. In other words, the term “higher” corresponds to a ratio [expression level in said test sample/expression level in said control sample] which is superior to 1 for said gene.

More precisely, if the ratio [expression level in said test sample/expression level in said control sample] is:
superior to 1.05, preferably to 1.1, more preferably to 1.14 for the LIZ gene;
superior to 1.05, preferably to 1.1, more preferably to 1.14 for the ELANE gene;
superior to 1.05, preferably to 1.1, more preferably to 1.15 for the G6PD gene;
superior to 1.1, preferably to 1.5, more preferably to 1.22 for the 6PGD gene;
superior to 1.1, preferably to 1.5, more preferably to 1.20 for the TKT gene; and/or
superior to 1.25, preferably to 1.3, more preferably to 1.34 for the CEACAM4 gene,
   then said human patient will have a long-term survival (i.e. a survival of at least 70 months, preferably 75 months and more preferably 80 months after the sample collection has been performed).

In a particular embodiment of the invention, the expression levels of the five genes G6PD (SEQ ID NO:2 or 3), 6PGD (SEQ ID NO:4), LILRB1 (SEQ ID NO:5 or 6 or 7 or 8), TKT (SEQ ID NO:9 or 10), CEACAM4 (SEQ ID NO:11) and ELANE (SEQ ID NO:23) are measured.

If the expression levels of the five genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) are higher in said test sample obtained from the patient, than those of the control sample, then said patient will live longer than 70 months, preferably 75 months and more preferably 80 months.

More precisely, if the ratio [expression level in said test sample/expression level in said control sample] is:
superior to 1.05, preferably to 1.1, more preferably to 1.15 for the G6PD gene;
superior to 1.05, preferably to 1.1, more preferably to 1.14 for the ELANE gene;
superior to 1.1, preferably to 1.5, more preferably to 1.22 for the 6PGD gene;
superior to 1.1, preferably to 1.5, more preferably to 1.20 for the TKT gene;
superior to 1.25, preferably to 1.3, more preferably to 1.34 for the CEACAM4 gene; and
inferior to 0.75, preferably to 0.7, and more preferably to 0.66 for the LILRB1 gene,
   then said human patient will have a long-term survival (i.e. a survival of at least 70 months, preferably 75 months and more preferably 80 months after the sample collection has been performed).

On the contrary, and as shown in the results below, if the expression levels of at least one of the five genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) are lower in said test sample obtained from the patient, than those of the control sample, then said patient will have a short-term survival, i.e., will live no more than 28 months, preferably 25 months and more preferably 21 months after the sample collection has been performed.

More precisely, if the expression levels of the five genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) are lower in said test sample obtained from the patient, than those of the control sample, then said patient will have a short-term survival, i.e., will live no more than 28 months, preferably 25 months and more preferably 21 months after the sample collection has been performed.

In a more particular embodiment, if the ratio[expression level in said test sample/expression level in said control sample] is
inferior to 1.14, preferably to 1.1, more preferably to 1.05 for the LIZ gene; and/or
inferior to 1.14, preferably to 1.1, more preferably to 1.05 for the ELANE gene; and/or
inferior to 1.15, preferably to 1.1, more preferably to 1.05 for the G6PD gene; and/or
inferior to 1.22, preferably to 1.5, more preferably to 1.1 for the 6PGD gene; and/or
inferior to 1.2, preferably to 1.5, more preferably to 1.1 for the TKT gene; and/or
inferior to 1.34, preferably to 1.3, more preferably to 1.25 for the CEACAM4 gene;
   then said human patient will have a short-term survival (i.e. a survival of at least maximally 28 months, preferably 25 months and more preferably 21 months after the sample collection has been performed).

In an even more preferred embodiment, if the ratio[expression level in said test sample/expression level in said control sample] is
inferior to 1.14, preferably to 1.1, more preferably to 1.05 for the ELANE gene; and
inferior to 1.15, preferably to 1.1, more preferably to 1.05 for the G6PD gene; and
inferior to 1.22, preferably to 1.5, more preferably to 1.1 for the 6PGD gene; and
inferior to 1.2, preferably to 1.5, more preferably to 1.1 for the TKT gene; and
inferior to 1.34, preferably to 1.3, more preferably to 1.25 for the CEACAM4 gene;
   then said human patient will have a short-term survival (i.e. a survival of at least maximally 28 months, preferably 25 months and more preferably 21 months after the sample collection has been performed).

In a second aspect of the invention, the present invention concerns a kit for in iv determining the prognosis of chronic myelomonocytic leukaemia in a human patient suffering thereof, comprising.

a) A reagent capable of specifically detecting the expression level of at least two genes chosen in the group consisting of: G6PD (SEQ ID NO:2 or 3), 6PGD (SEQ ID NO:4), ELANE (SEQ ID NO:23), TKT (SEQ ID NO:9 or 10) and CEACAM4 (SEQ ID NO:11), and
b) Instructions for using said kit for determining the prognosis of chronic myelomonocytic leukaemia in said human patient.

The kit can also comprise a reagent capable of specifically detecting the expression level of the LYZ gene of SEQ ID NO:1.

By “reagent capable of specifically detecting the expression level of” is meant a reagent specifically intented for the specific determination of said expression levels, either on the transcription (RNA) or on the translation (proteic) levels. This definition excludes generic reagents useful for the determination of the expression level of any gene, such as taq polymerase or an amplification buffer, although such reagents may also be included in a kit according to the invention.

In any kit for the in vitro prognosis of the survival of CMML suffering patients according to the invention, the reagent(s) for specifically detecting the expression level of the genes comprising, or consisting of, the 6 genes from Table 1 or homologous thereof, preferably include specific amplification primers and/or probes for the specific quantitative amplification of transcripts of genes of Table 1, and/or a nucleic microarray for the detection of genes of Table 1. The determination of the expression levels may thus be performed using quantitative PCR and/or a nucleic microarray, preferably an oligonucleotide microarray.

In addition, the instructions for the determination of the survival of CMML suffering patients preferably include at least one reference expression profile, or at least one reference sample for obtaining a reference expression profile. Preferably, the determination of the patient survival is carried out by comparison with the test sample and the reference sample as described above.

In another aspect, the invention is also directed to a nucleic acid microarray comprising or consisting of nucleic acids specific for the 6 genes from Table 1 or homologous thereof. Said nucleic acid microarray may comprise additional nucleic acids specific for genes other genes. Advantageously, said microarray consists of nucleic acids specific for the 6 genes of Table 1 above. In a preferred embodiment, said nucleic acid microarray is therefore an oligonucleotide microarray comprising or consisting of oligonucleotides specific for the 6 genes from Table 1.

As mentioned above, the man skilled in the art perfectly knows how to design “oligonucleotides specific for a gene” in view of its gene accession number.

All the embodiments concerning nucleic acid microarrays and methods of preparing them have been developed above defacto apply to the nucleic acid microarray of the invention.

In another aspect, the present invention also relates to a mRNA prognostic signature for predicting outcome of a patient suffering from chronic myelomonocytic leukaemia, independently from other factors, comprising one or more up-regulated mRNAs of the genes chosen in the group consisting of the LYZ (SEQ ID NO:1), G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) genes or homologous thereof, as compared with mRNA of same genes expressed in normal cells.

In a preferred embodiment, the expression level of at least three, preferably four, more preferably five genes chosen in the group consisting of the ELANE (SEQ ID NO:23), G6PD (SEQ ID NO:2 or 3), 6PGD (SEQ ID NO:4), TKT (SEQ ID NO:9 or 10) and CEACAM4 (SEQ ID NO:11) genes or homologous thereof is measured in the method of the invention.

All the embodiments concerning said genes and methods of assessing there expression level that have been developed above de facto apply to said mRNA prognostic signature.

Preferably, said expression levels of said genes are measured in PBMC cells, obtained either from the patient, or from a reference healthy human subject.

Finally, the present invention also relates to a method for determining if patients suffering from chronic myelomonocytic leukaemia will have a short-term survival or a long-term survival comprising the steps of:

a) obtaining a test sample from said human patient,
b) determining the expression level of the at least two genes chosen in the group consisting of: the LYZ (SEQ ID NO:1), G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) genes or homologous thereof in said test sample, and
c) applying a predictive model for determining if said patient will have a short-term survival or a long-term survival.

This method enables for example to identify and select the patients belonging to each group (short- or long-term survival patient groups) which can be used in particular clinical trials. It can advantageously be used as pharmacogenomic information in companion diagnostic tests. These pharmacogenomic biomarkers can help differentiate patient into responder and non responder groups, which can help estimate drug effectiveness, avoid toxicity and adverse effects, increase drug safety and adjust drug dosage and are therefore encouraged by several health Authorities.

As a matter of fact, labelling drug has become more difficult within the last 10 years. During the 3 last months Avastin® (Roche Pharmaceutical) has been recalled for breast cancer application and Aflibercept (Sanofi-Aventis) fell in late clinical trials Phase III for lung cancer application. Therefore drug approval agencies, including FDA and EMEA, are encouraging greater use of biomarker and diagnostic in drug development and prescribing decision. This encouragement and guidance has taken several forms (1), (2), including the last Guidance for Industry Clincal Pharmacogenomics: premarketing Education in early Phase Clincal Studies (3) issued in February 2011. Proof of concept of the use in clinical studies as well as in prescribing decision of such biomarkers has been achieved for Big Pharma like Genetech Roche with Trastuzumab (Her2), a molecule labelled and associated with Companion Diagnostic. About 10% of labels for drugs approved by the FDA now contain pharmacogenomic information. Such pharmacogenomic biomarkers can thus help to increase the chance to be approved by health Authorities. More conclusively, both FDA and EMEA now require that biomarker testing be performed prior to prescribing certain drugs.

For patient suffering from chronic myelomonocytic leukaemia, the method of the invention enables for example to select those requiring an aggressive treatment (such as bone marrow transplant) from those requiring “only” supportive care (administration of blood product support and/or hematopoietic growth factors).

More precisely, it is considered that short-term survival patient groups will be preferentially included in clinical trials involving bone marrow transplantation (stem cell transplantation), or aggressive chemotherapy, for example with hypomethylating agents such as 5-azacytidine, decitabine, or lenalidomide.

On the contrary, long-term survival patient groups will be preferentially included in clinical trials involving iron uptake, or red blood cell transfusion (optionally with a chelation therapy to avoid iron overload).

In a preferred embodiment, said predictive model is reduced to practice by calculating an index as follows:

First, the expression levels of each of the five genes are measured in a patient sample and are compared to the reference expression. A ratio is calculated, leading to the calculation of a “fold change”. This fold change is compared to cut-off values, and patients are then dichotomised (+1 or −1 for gene value under or below the significant cut-off) for each significant gene and pondered by the beta-coefficient of each genes (which have been calculated from Kaplan-Meier analysis).

In a preferred embodiment, the following cut-offs values and Beta-coefficients are used:

| | Fold | Cut- | Dichotomisation: | Beta-coefficient |
|---|---|---|---|---|
| Gene name | change | off | D = | (β) |
| ELANE | b | 3.40 | +1 if b > 3.40; −1 | 2.01784191521554 |
| | | | if b ≦ 3.40 | |
| G6PD | c | 1.15 | +1 if c > 1.15; −1 | 1.28224877578792 |
| | | | if c ≦ 1.15 | |
| TKT | d | 1.2 | +1 if d > 1.2; −1 | 1.35358578043486 |
| | | | if d ≦ 1.2 | |
| PGD | e | 1.22 | +1 if e > 1.22; −1 | 1.71153730912409 |
| | | | if e ≦ 1.22 | |
| CEACAM4 | f | 1.34 | +1 if f > 1.34; −1 | 2.0942792881 |
| | | | if f ≦ 1.34 | |

Then, for each patient, the index was calculated by the sum of the dichotomised value pondered by the beta-coefficient of each gene:

I=D_{LYZ}×β_{LYZ}+D_{LILRB1}×β_{LILRB1}+D_{G6PD}×β_{G6PD}+D_{TKT}×β_{TKT}+D_{PGD}×β_{PGD}+D_{CEACAM4}×β_{CEACAM4}

If the calculated index I is superior to 1, then short-term survival is to be prognosed for said patient.

If the calculated index I is inferior or equal to 1, then long-term survival is to be prognosed for said patient.

Preferably, in this aspect of the invention, the expression level of all the five genes ELANE (SEQ ID NO:23), G6PD (SEQ ID NO:2 or 3), 6PGD (SEQ ID NO:4), TKT (SEQ ID NO:9 or 10) and CEACAM4 (SEQ ID NO:11) is measured.

More preferably, said predictive model comprises:
i) calculating the ratio between the expression level of the said genes in said test sample and the expression level of the same genes in a control sample of a known healthy human subject,
ii) comparing said ratio with cut-offs values for each gene and determining the dichotomisation factors for each gene.
iii) pondering said dichotomisation factors by predetermined beta-coefficient for each genes, and
iv) calculating an index I which is the sum of said dichotomised factors pondered by said beta-coefficients of said genes for said patient:
   In other words, said index I is calculated as follows:
   I=D_{LYZ}×β_{LYZ}+D_{LILRB1}×β_{LILRB1}+D_{G6PD}×β_{G6PD}+D_{TKT}×β_{TKT}+D_{PGD}+β_{PGD}+D_{CEACAM4}×β_{CEACAM4}, D being the dichotomisation factor of each gene and P the P coefficient of each gene.

The calculated index I is then compared to the value 1 so as to determine if said patient will have long- or short-term survival:

If the calculated index I is superior to 1, then short-term survival is to be prognosed for said patient.

If the calculated index I is inferior or equal to 1, then long-term survival is to be prognosed for said patient.

Having generally described this invention, a further understanding of characteristics and advantages of the invention can be obtained by reference to certain specific examples and figures which are provided herein for purposes of illustration only and are not intended to be limiting unless otherwise specified.

### EXAMPLES

### Introduction

Chronic myelomonocytic leukaemia (CMML) is a clonal hematopoietic stem cell disorder frequently seen in the elderly. First considered as a myelodysplastic disease in the French American British (FAB) classification (Bennett et al., 1994), CMML was reclassified by the World Health Organization (WHO) as a myelodysplastic/myeloproliferative neoplasm (MDS/MPN) (Jaffe et al., 2001). This reclassification underlines the heterogeneity of CMML in diagnosis and prognosis. Despite of this heterogeneity, the diagnosis of CMML is definitely straightforward in the presence of a combination of persistent blood monocytosis, fewer than 20% blasts in peripheral blood and bone marrow, absence of BCR-ABL1 fusion gene and dysplasia in one or more cell lines (Vardiman et al, 2002; Orazi & Germing, 2008). According to WHO criteria, blasts include myeloblasts, monoblasts and promonocytes. The myeloid compartment is frequently associated with cytogenetic abnormalities that help to confirm the CMML diagnosis, but none are specific (Reiter et al, 2009).

In order to characterize factors predicting the course of the disease, recent data based on mutation identification have been reported, among them RAS and TET2 are the most frequently affected genes. Twenty two percent of patients exhibit point mutations of RAS genes (NRAS, KRA) at diagnosis or during the disease course and as many as 50% present TET2 mutations (Ricci et al., 2010; Kosmider et al., 2009). With respect to clinical data, Kosmider et al. suggest that the prevalence of TET2 mutations is higher in CMML than in any other myeloid disease and is associated with a trend to a lower overall survival rate. On the other hand, by applying next-generation sequencing (NGS) technology, two recent reports detected frequent aberrations in the TET2 gene in CMML cases and related it to better outcome (Kohlmann et al., 2010; Grossmann at al, 2011).

Currently, no reliable molecular prognostic markers are available with an easy technology in CMML in spite of the recent WHO reclassification. The difficulty of the clinical classification and the variable risk of progression to acute myeloid leukemia (AML) remain the major problems for physicians.

In light of these issues, we have chosen to perform gene expression profiling (GEP) as molecular studies in CMML using this approach, have not been extensively explored (Theilgaard-Monch et al., 2011). The aim of our study was to identify molecular predictors, from 32 CMML peripheral blood mononuclear cells (PBMC), associated with better survival and to validate its performance in an independent test set of 21 CMML samples. The present work shows that GEP has a prognosis potential in CMML and could help improving the classification of the disease.

### Design and Methods

### Patients and Control Samples

CMML diagnosis was defined according to the World Health Organization (WHO) criteria, as previously published (Reiter et al., 2009; Orazi & Germing, 2008; Vardiman et al., 2002). The patients signed informed consent to participation in the study in accordance with the Declaration of Helsinki. The study was approved by the ethic board of Nímes University. PBMCs were collected in the Centre Hospitalier Universitaire (CHU) of Nímes from 32 patients who were newly diagnosed. All samples in this study were obtained from untreated patients at the time of diagnosis. For 14 patients, paired material at presentation and at different periods of follow-up was also available for gene expression analyses. Sixteen blood samples of acute myeloid leukaemia (AML) and two samples of proliferative and differentiated U937 leukaemia cells, cultured as previously described (Piquemal et al., 2002), were also included in the analyses. AML samples include 4 de now and 12 secondary AML (transformed CMML). Control samples of PBMC obtained from three healthy donors were used as reference.

### Molecular Markers Screening

Genes were selected from transcriptomic data established by SAGE methodology from acute myeloid leukaemia models, normal polymorphonuclear and monocytic cells (Piquemal et al., 2002; Bertrand at al., 2004; Quire et al., 2007; Rivals et al., 2007). Differential gene expression analyses were performed as previously described (Piquemal et al., 2002). SAGE libraries data are available at GEO (http://www.ncbi.nlm.nih.gov/geo/) under accession number GSM32698: untreated U937 cell line; GSM32699: differentiated U937 cell line; GSM151619: untreated NB4 cell line; GSM151622 differentiated NB4 cell line. The SAGE libraries were described in Rivals et al. and Bertrand et al. for normal monocytes and granulocytes, respectively (Rivals et al., 2007; Bertrand a al, 2004). By mining the SAGE data, 92 transcripts showing significant variation following myeloid cell differentiation and 1 calibration marker (RPS19) were selected for high-throughput real-time polymerase chain reaction (PCR) analysis. The listing of the 93 genes is provided in supplementary data. They correspond to transcripts over-expressed in leukaemia differentiated cells, cell cycle genes and transcripts already known as cancer-related genes (Piquemal et al., 2002; Quéré et al., 2007). We used also Affymetrix data of 21 CMML samples from the Microarray Innovations in Leukaemia (MILE) study (Haferlach et al., 2010). All samples were obtained from untreated patients at the time of diagnosis. These data are publicly available via GEO under accession number GSE13204. Information on survival and clinical parameters were provided by Pr Mills's group.

### RNA Extraction, Reverse Transcription, and High-Throughout Real-Time PCR

RNA was extracted with RNeasy Qiagen kit. RNA quality was monitored and quantified using the 2100-Bioanalyzer (Agilent Technologies, Waldronn, Germany). Reverse transcription was performed with random primers (High-capacity cDNA Archive kit; Applied Biosystems, Courtaboeuf, France) using 1 μg total RNA. PCR analyses were performed on microfluidic cards with 100 ng of cDNA, using the TaqMan® Gene Expression Assays and the ABI7900HT system (Université de Limoges Q-PCR facility). Analysis of the relative quantity gene expression (RQ) data was performed using the 2^{-ΔΔCt} method (Livak & Schmittgen, 2001). Transcriptional modulation (log₁₀ RQ) was calculated using data from normal PBMCs as reference. Data were collected and analysed with Sequence Detector Software (SDS2.2; Applied Biosystems). Similar results were obtained from relative quantity gene expression comparisons using the 3 calibrator genes. For the final normalization, RPS19 was selected. The accuracy of the technology was validated by testing the reliability of SAGE and the high-throughput real-time PCR. Among the differentially expressed markers selected from the SAGE data (P≦0.01), 95% displayed significant modulation when tested on microfluidic cards. Standard error (SE) was measured using U937 samples already tested in a separate study. Paired samples from 26 patients were tested to evaluate the reproducibility of our method. In the unsupervised hierarchical cluster, each sample and its duplicate came out together in the same subtype.

### Statistical Analysis

Genes with no measured expression in all samples were discarded. A total of 93 genes were selected for unsupervised analysis. Hierarchical clusters were performed with the Cluster and Treeview softwares from Eisen et al (Eisen et al., 1998). Gene expression data was analysed with SAM (Significance Analysis of Microarrays) software with a 1000-permutations adjustment (Cui & Churchill, 2003). For each selected gene, the patients' samples were ordered by low to high expression values. For each increasing signal position in this scale, the overall survival difference between patients having a lower or equal versus a higher signal was assessed using a log-rank test with the Maxstat package used in R software (http://cran.r-project.org/). Overall survival of subgroups of patients was compared with the log-rank test and survival curves computed with the Kaplan-Meïer method (R software; survival package). Benjamini and Hochberg Multiple Testing correction was used to select the strongest genes associated with the overall survival (Camargo et al, 2008). At rank one, this within-probe adjustment is realized by multiplying the maximum P-value by the number of calculated positions. Genes with P value>0.05 were discarded (Carlin & Chib, 1995). For the index computation, first patients were dichotomised (+1 or −1 for gene value under or below the significant cut-off) for each significant gene and pondered by the beta-coefficient (issued from Kaplan-Meïer analysis). Then, for each patient, the index was calculated by the sum of the dichotomised value pondered by the beta-coefficient of each gene (Kassambara et al., 2011). Statistical comparisons were done with Mann-Whitney, Chi-square, or unpaired or paired Student's t tests.

The networks were generated through the use of Ingenuity Pathways Analysis (Ingenuity Systems, www.ingenuity.com). A data set containing gene identifiers and corresponding expression values was uploaded into the application. Each gene identifier was mapped to its corresponding gene object in the Ingenuity Pathways Knowledge Base. These genes, called focus genes, were overlaid onto a global molecular network developed from information contained in the Ingenuity Pathways Knowledge Base. Networks of these focus genes were then algorithmically generated based on their connectivity. Gene expression data were extracted from the Oncomine Cancer Microarray database (http://www.oncomine.org) (Rhodes et al., 2004) and the Amazonia database (http://amazonia.montp.inserm.fr) (Le Carrour et al., 2010).

### Results

### Patients

A total of 32 CMML patients including 21 males (66%) and 11 females (34%) were studied. Their main clinical and haematological characteristics are shown in Table I. We had same proportions of different clinical parameters as previously described (Such et al; 2011). Median age was 76 years (range 45-86). According to FAB criteria, 15 patients (47%) had MD-CMML and 17 patients (53%) had MP-CMML. According to WHO classification, 27 patients (90%) were diagnosed as having CMML-1 and 3 patients (10%) as having CMML-2. Karyotype was normal in 20 patients (63%) and abnormal in 4 patients (13%); data were not available for 8 patients (25%). Among cytogenetic aberrations, we find one patient with trisomy 8, one patient with monosomy 7, one patient with loss of the Y chromosome and one patient with other anomalies. Five patients developed acute myeloid leukaemia of which three showed an abnormal karyotype. There were no significant differences in blast proportion in patients' bone marrow.

### Gene Expression-Based Analyses Defines Two Subsets of CMML Patients

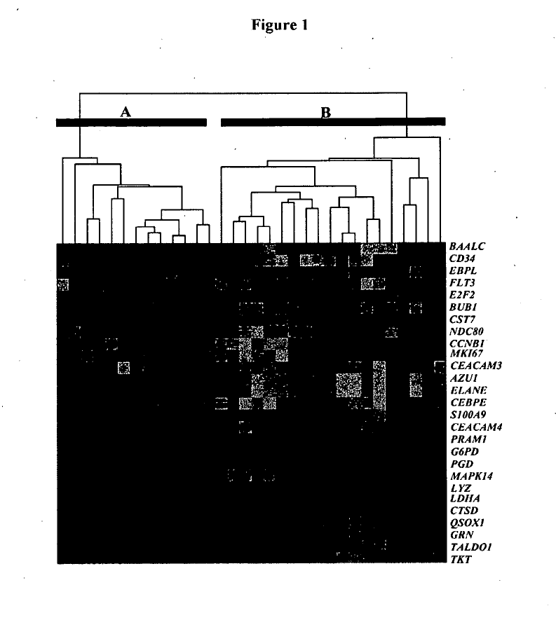

We undertook a comparison study of gene expression variation between different clinical samples. Gene expression data were generated from PBMC cDNA obtained for 32 CMML patients and their paired samples, 4 de now AML patients and 2 samples of proliferative or differentiated U937 cells using microfluidic low density arrays. Using an unsupervised hierarchical clustering approach, two main groups of samples were defined: G1 and G2. De now and secondary AML and U937 samples came out together in the G1 group, while all CMML samples clustered in the G2 group, which was subdivided into two subgroups: G2A and G2B. In order to select genes which could highly discriminate between the identified subgroups, we employed a supervised approach using Significance Analysis of Microarrays (SAM) tool. Twenty-eight genes passed SAM analysis with a false discovery rate (FDR)<5%. These genes were selected as a ‘predictor set’ for survival. They enabled the characterization of two categories of patients with different gene signatures (FIG. 1). We next investigated which of them are known to interact biologically by carrying out pathway analysis using the Ingenuity Pathway Analysis (IPA) tool. Twenty-two genes mapped to genetic networks and two networks were found to be highly significant (51 and 18 as respective scores). They were mainly associated with cell cycle, DNA replication, and cellular growth and proliferation.

### ‘Survival Index’ Scoring and Biological Significance

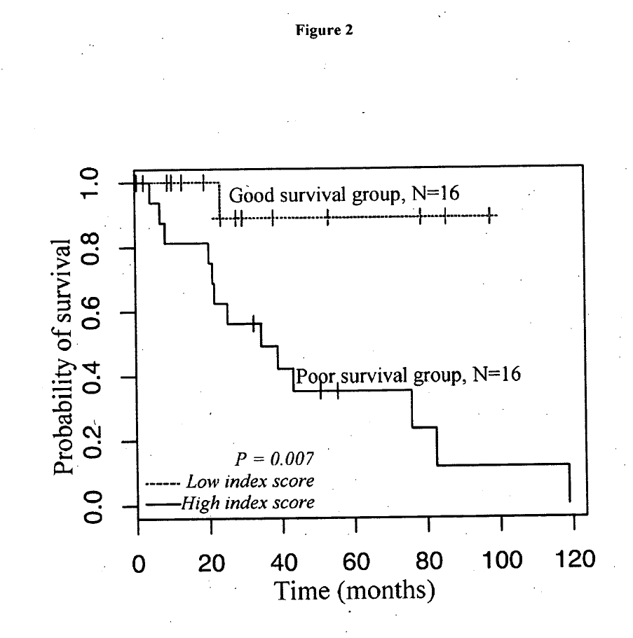

In order to stringently identify a gene signature predictive of survival, we aimed to construct a ‘prognosis index’ which can separate categories of patients with different survival. To do so, overall survival (OS) curves were plotted for each gene in the ‘predictor set’ and P-values were corrected by Benjamini and Hochberg multiple testing correction. Five genes showed a significant bad prognostic value: G6PD; Glucose-6-phosphate dehydrogenase, PGD; 6-phosphogluconate dehydrogenase; TKT; Transketolase, ELANE; Neutrophil elastase and CEACAM4; Carcinoembryonic antigen-related cell adhesion molecule 4. We computed the ‘prognosis index’ by combining the prognostic information of the five selected genes as described in materials and methods. OS curve was plotted (FIG. 2). Patients were distributed between two groups: good (dotted grey) and poor survival (black) with 50% of patients in each group. As shown in FIG. 2, OS was significantly increased in patients with low survival index score. The 10-year OS was 94% in the good prognosis group versus 19% in the poor prognosis group.

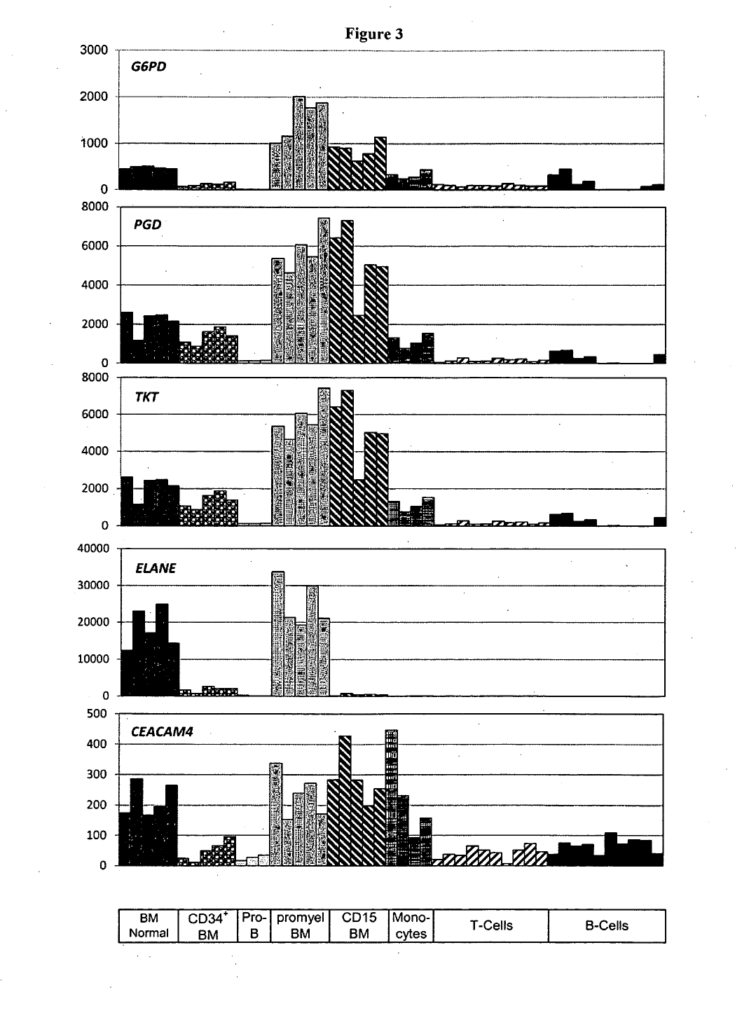

We compared the expression of our 5 genes in a panel of 16 cancer types to their normal counterparts using the Oncomine Cancer Microarray database, a publicly available gene expression data (Table II). Interestingly, the 5 genes are expressed at least in ⅓ haematological cancers and 4/13 solid tumours. TKT was found to be over-expressed in leukaemia, lymphoma, myeloma and expressed in 10/13 solid tumours. When comparing their expression profiles in various normal haematological tissues using the public microarray database Amazonia, TKT, G6PD, PGD, ELANE and CEACAM4 displayed a myeloid phenotype and were expressed in normal bone marrow (FIG. 3). ELANE shows a promyelocytic restricted pattern, as TKT, G6PD, PGD and CEACAM4 are also expressed in immature and differentiated granulocytes and in monocyte populations.

Index Association with Clinical Characteristics and Validation

We investigated association of the index survival groups obtained with clinical and biological characteristics. We observed no specific pattern with age, gender and cytogenetic abnormalities. As shown in table III, there were neither association with FAB, WHO and IPSS (International Prognosis Scoring System) classification systems. Anyhow, with respect to clinical data, no significant prognostic difference was detectable for MD-CMML and MP-CMML categories (P=0.39, data not shown). Yet, due the limited number of CMML-2 compared to CMML-1 cases, we did not separate the cohort into these two categories in subsequent analyses.

With regard to treatment and AML transformation, 76% of treated patients were found in the group of worse survival. This correlated with progression, as all AML-transformed patients were also included in this category. This observation could suggest a more aggressive disease that progresses over time.

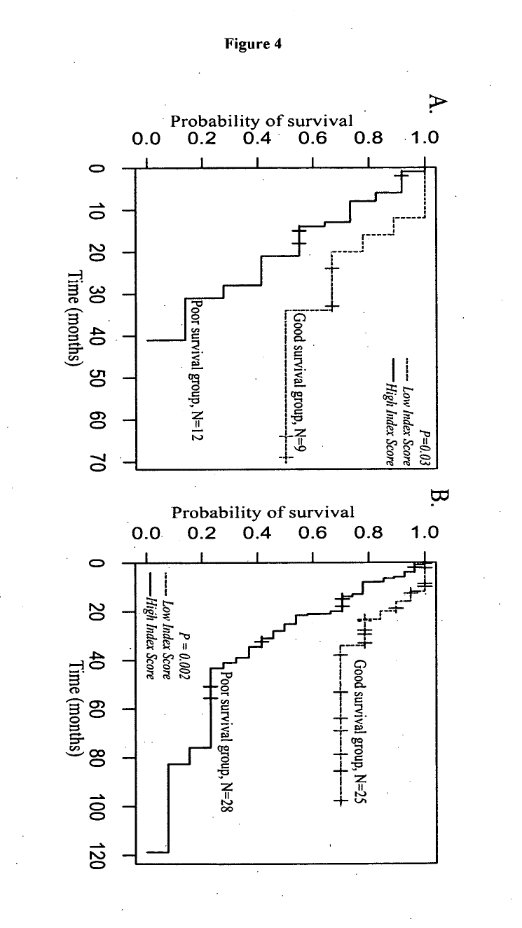

Assuming that our ‘prognosis index’ is able to discriminate between different clinical samples, we sought to demonstrate its robustness and prognostic independence in a new cohort of 21 CMML patients that were included in the MILE study (Haferlach et al., 2010). Briefly, this cohort consists of 15 patients (71%) with normal karyotype and 6 patients (29%) with abnormal karyotype. Median age was 74.7 years. IPSS varied between favourable (11 patients, 52%) and intermediate-1 (9 patients, 48%). 4 patients (19%) evolved to AML. We performed an index based survival analysis using the new cohort. Contrary to our gene expression data obtained from TaqMan low density arrays, we used here HG-U133Plus2.0; Affymetrix data. Despite that, we successfully identified two categories of patients with significant outcomes (P=0.03) (FIG. 4A). Samples were equally distributed in each group. Similarly, we observed no specific correlation between the obtained classification of samples and other clinical and biological characteristics. When adding the two cohorts together (53 patients in total) (FIG. 4B), the statistical prognostic value was yet increased (P=0.002). These results shows that our five gene based ‘prognosis index’ could be adapted to other cohorts of CMML with distinct types of gene expression data. It could be a powerful tool to predict clinical outcome and to discover novel subclasses for this malignancy.

### Discussion

In haematological malignancies, GEP allowed for detecting new biologically and prognostically relevant subtypes despite the genetic heterogeneity of the disease (Moreaux et al., 2011; Wouters et al., 2009; Bresolin et al., 2010). The objective of our study was to select genetic markers which could be proposed as new tool for prognosis in CMML. Using microfluidic low density arrays, we profiled a series of 32 untreated CMML patients at diagnosis. By supervised analysis, we identified 28 out of the 93 selected genes. We then established a five-gene prognostic index potentially more easily applicable in daily clinical practice. Using this index, we classified patients, independently from classical prognostic features, in two groups with different clinical outcome: a good class with 10-year OS of 94%, and a poor class with 10-year OS of 19%. Importantly, the strength and prognostic independent value of our survival index was successfully checked on a validation cohort of 21 CMML patients with data obtained from Affymetrix microarrays. All together, we demonstrated the usefulness of GEP prognostic in CMML regardless of the quantitative gene expression method.

The significant networks we identified as related to cell cycle, DNA replication, and cellular growth and proliferation corroborated with published data. Alterations in biological processes that contribute to an adaptation of tumour cells and an increase of their aggressiveness were also observed. Among our prognostic predictors, we found G6PD, TKT and PGD which displays a significant function in glycolysis by regulating the pentose phosphate pathway. They favour the production of ribose which is essential for RNA and DNA synthesis in rapidly growing cells. Deregulation of this metabolic pathway radically alters G6PD, TKT and PGD genes promoting tumour cell proliferation and poor prognosis; hence their elevated levels of expression and activity in breast, colon and various other types of cancer (Baba et al., 1989; Toyokuni et al., 1995; Furuta et al., 2010). CEACAM4, a carcinoembryonic antigen (CEA) family member, is uniquely expressed on primary human granulocytes (Schmitter et al., 2007). CEACAM proteins are well-known markers associated with progression of colorectal tumours. Interestingly, the Oncomine Cancer Microarray database confirms that four out of our five outcome predictors are over expressed in haematological cancers and solid tumours. TKT was the more frequently involved as it was found to be over-expressed in leukaemia, lymphoma, myeloma and major solid tumours.

In the same way, the molecular markers identified in the present study could facilitate the identification of key pathways and abnormal cell subtypes involved in CMML. When comparing expression profiles of the five genes in various haematological tissues, all of them displayed a myeloid phenotype as they are mainly expressed in immature and differentiated granulocytes. ELANE shows the more restricted phenotype as it's exclusively expressed in promyelocytic cells. Recently, Droin et al. (2010) explored the cellular heterogeneity of the leukaemia clone and underlined the presence of immature dysplastic granulocytes in the peripheral blood. These cells, clearly distinct from CD14⁺ monocytes, belong to the tumoral population and highly express CEBPE and GFI1, two transcription factors involved in the myeloid lineage that controls ELANE gene expression, one of the detected molecular markers. It's not clear if this granulocytic immature population is present in all CMML patients but in the present study, it's noteworthy that high expression levels of promyelocytic and immature granulocyte markers with cell cycle characteristics correlate with a poor prognostic. It would be interesting to determine if the molecular predictors correlate with the presence of distinct leukemia cell populations in the peripheral blood with specific proliferative status.

In conclusion, we have developed, and validated in two independent series of samples, a five-gene index associated with survival. The heterogeneity of the disease reflected by the current classification system doesn't sufficiently contribute to stratify high risk patients. As already described from microarray data analysis of myelodysplastic syndromes (Mills et al., 2009), our data demonstrated the prognostic potential of GEP in CMML and revealed the heterogeneity of this disorder that would be essential for therapeutic proposals. Indeed, the poor survival profile seems to correlate with a more aggressive disease as the group included most of patients receiving a treatment and those presenting a high risk of AML transformation. Conversely, the fact that the favourable group is mainly characterised by the absence of treatment, could reflect a more indolent form. Furthermore, a better understanding of the implication of these genes in CMML and their power in respect to prognosis could be of clinical interest for physicians.

TABLE I
Clinical, haematological and molecular features of the 32 CMML patients

| | | Peripheral Blood | | Bone Marrow | | | | | | Progression | Time to |
|---|---|---|---|---|---|---|---|---|---|---|---|
| | sex/age | WBC | Monocytes | Blarts | Monocytes | | Treatment at sampling | | | after | progression |
| N^{o} | (years) | (G/I) | (G/I) | (%) | (%) | Karyotype | Chemo. | Transfu. | CSF | sampling | (months) |
| 1 | M/83 | 17.4 | 1.65 | ND | ND | ND | no | no | no | no | |
| 2 | M/45 | 30.0 | 3.9 | 7 | 14 | 45 XY, −7 | no | yes | yes | AML 4 | 13 |
| 3 | M/66 | 11.8 | 1.9 | 1 | 4 | 46 XY | no | no | no | no | |
| 4 | F/84 | 2.8 | 1.1 | 3 | 6 | ND | no | yes | no | no | |
| 5 | M/82 | 12.9 | 2.7 | 1 | 12 | ND | no | yes | no | no | |
| 6 | M/62 | 6.2 | 1.9 | 1 | 1 | 46 XY | no | yes | no | no | |
| 7 | M/68 | 18.3 | 6.6 | 3 | 25 | 46 XY | yes | yes | no | no | |
| 8 | M/85 | 13.3 | 6.7 | 11 | 28 | ND | no | no | no | no | |
| 9 | M/62 | 10.3 | 3.4 | 4 | 15 | 46 XY | yes | yes | no | AML1 | 48 |
| 10 | M/73 | 7.0 | 2.2 | 2 | 6 | 46 XY | no | yes | no | no | |
| 11 | M/73 | 4.6 | 1.4 | 4 | 18 | ND | no | yes | no | no | |
| 12 | M/73 | 4.2 | 0.9 | 3 | 14 | 45, −Y | no | no | no | no | |
| 13 | M/68 | 23.3 | 4.1 | 2 | 8 | 46 XY | no | no | no | no | |
| 14 | F/83 | 12.1 | 2.1 | 0 | 10 | 46 XX | no | no | no | no | |
| 15 | M/80 | 9.4 | 2.5 | ND | ND | ND | no | no | no | no | |
| 16 | M/81 | 4.0 | 1.0 | 2 | 6 | 46 XY | no | no | no | no | |
| 17 | F/85 | 6.6 | 1.8 | 1 | 7 | ND | no | yes | yes | no | |
| 18 | M/69 | 37.8 | 6.4 | 1 | 5 | 46 XY | no | yes | no | no | |
| 19 | M/80 | 10.5 | 3.0 | 2 | 7 | 46 XY | no | yes | no | AML 4 | 8 |
| 20 | M/55 | 19.0 | 1.7 | 2 | 4 | 46 XY | no | no | no | no | |
| 21 | M/57 | 5.9 | 2.9 | 2 | 9 | 46 XY | no | no | no | no | |
| 22 | F/79 | 30.6 | 2.8 | 1 | 1 | 46 XX | no | no | no | no | |
| 23 | F/86 | 4.4 | 1.2 | 2 | 29 | 46 XX | yes | no | yes | no | |
| 24 | M/76 | 15.8 | 3.8 | 11 | 10 | 46 XY | no | yes | no | no | |
| 25 | F/65 | 11.1 | 2.2 | 1 | 10 | 46 XX | no | on | no | no | |
| 26 | F/82 | 6.6 | 1.4 | 1 | 9 | 46 XX | no | no | no | no | |
| 27 | M/80 | 73.1 | 13.2 | 2 | 11 | 46 XY, t(13.22), del(13) | yes | yes | no | AML 4 | 32 |
| 28 | M/81 | 5.4 | 1.6 | 1 | 11 | 46 XY | no | yes | no | no | |
| 29 | F/73 | 9.9 | 1.9 | 5 | 3 | ND | yes | yes | no | no | |
| 30 | F/79 | 6.9 | 1.7 | 4 | 14 | 47XX, +8 | yes | yes | no | AML 4 | 17 |
| 31 | F/76 | 13.7 | 4.5 | 1 | 6 | 46 XX | no | no | no | no | |
| 32 | F/75 | 6.9 | 2.1 | 8 | 12 | 46 XX | no | no | no | no | |

TABLE II
Expression of genes encoding TKT, G6PD, PGD, ELANE and CEACAM4
in human cancer samples in comparison to their normal counterparts.
Expression data were obtained from the Oncomine Cancer Microarray database.
Genes which were over- and under-expressed in cancer cell samples
in comparison with their normal counterpart are indicated in this table.

| | | Genes over- and under-expressed in cancer samples | | | | |
|---|---|---|---|---|---|---|
| | Cancer sample | in comparison to their normal tissue counterpart | | | | |
| | type | TKT | G6PD | PGD | ELANE | CEACAM4 |
| Haematological | Leukaemia | Up | Up | Down | Up | Down |
| cancer | Lymphoma | Up | — | Up | — | — |
| | Myeloma | Up | — | — | — | — |
| Solid Tumours | Bladder cancer | Up | — | Up | — | — |
| | Brain cancer | Down | — | — | Up | — |
| | Breast cancer | Down | — | Down | — | — |
| | Colorectal | Up | Up | Up | Down | Down |
| | cancer | | | | | |
| | Gastric cancer | Up | — | — | Down | — |
| | Liver cancer | Up | Up | — | — | — |
| | Lung cancer | Up | — | Up | — | — |
| | Melanoma | Up | Up | — | — | — |
| | Ovarian cancer | Up | Up | — | Up | — |
| | Pancreatic | Down | Up | — | — | Down |
| | cancer | | | | | |
| | Prostate cancer | Up | — | — | — | — |
| | Renal cancer | Up | Up | — | Up | Down |
| | Testicular cancer | Up | — | Up | — | — |

TABLE III
Correlations between results of the ‘Survival Index’ classification
and patients' clinical and biological characteristics

| | Good survival | | Poor survival | |
|---|---|---|---|---|
| | group (n = 16) | | group (n = 16) | |
| Characteristic | No. | % | No. | % |
| Median Age at Diagnosis | 73.3 | | 74.6 | |
| Gender | | | | |
| Female/Male | 5/11 | 31/69 | 6/10 | 37/63 |
| Cytogenetic abnormalities | | | | |
| (data available for 24 patients) | | | | |
| Normal Karyotype | 10 | 91 | 10 | 77 |
| Abnormal karyotype | 1 | 9 | 3 | 23 |
| FAB classification | | | | |
| (data available for 32 patients) | | | | |
| MD-CMML | 7 | 44 | 8 | 50 |
| MP-CMML | 9 | 56 | 8 | 50 |
| WHO classification | | | | |
| (data available for 30 patients) | | | | |
| CMML-1 | 12 | 86 | 15 | 94 |
| CMML-2 | 2 | 14 | 1 | 6 |
| IPSS classification | | | | |
| (data available for 24 patients) | | | | |
| Favourable | 9 | 50 | 9 | 50 |
| Int-1 | 1 | 25 | 3 | 75 |
| Int-2 | 1 | 50 | 1 | 50 |
| Treatment during follow-up | | | | |
| (data available for 32 patients) | | | | |
| Chemotherapy | 1 | 17 | 5 | 83 |
| Supportive treatment (Transf & HGF) | 3 | 27 | 8 | 73 |
| Alt treatments | 4 | 24 | 13 | 76 |
| AML-transformation | 0 | 0 | 5 | 100 |
| (data available for 32 patients) | | | | |

Abbreviations: FAB, French-American-British classification; MD, Myelodysplastic; MP, Myeloproliferative; WHO, World Health Organisation; IPSS, International Prognosis Scoring System; Int-1, Intermediate 1; Int-2, Intermediate 2; HGF, Hematopoietic Growth Factors.

### BIBLIOGRAPHY

Baba M et al., Int. J. Cancer 1989; 43(5):892-5
Benjamini Y, 1995, Journal of the royal statistical society B57, 289-300
Bennett, J. M., (1994) British Journal of Haematology, 87, 746-754.
Bertrand G et al. J Immunol Methods. 2004; 292(1-2):43-58.
Bonafoux B and Commes T, Methods Mol Biol. 2009; 496:299-311
Bresolin, S., (2010) Journal of Clincal Oncology, 28, 1919-1927.
Camargo A et al., Source Code Biol Med, 2008; 3:15
Carlin & Chib (1995) Journal of the Royal Statistical Society, Series B57, 473-484.
Cui X, et Churchill G A, Genome Biol. 2003; 4(4):210
Droin, N., (2010). Blood, 115, 78-88.
Eckel-Passow J E et al. Cancer Res. 2005 Apr. 15; 65(8):2985-9
Eisen M B, et al. Proc Natl Acad Sci USA. 1998; 95:14863-14868
Furuta E et al., Biochimica et Biophysica Acta, 2010; 1805(2):141-52
Grossmann, V., (2011). Leukemia, 25, 877-879.
Haab B B. Mol Cell Proteomeics. 2005 April; 4(4):377-83
Haferlach, T., (2010). Journal of Clincal Oncology, 28, 2529-2537.
Hall D A et al. Meth Ageing Dev. 2007 January; 128(1):161-7
Jaffe, E. S., (2001) Lyon: IARC Press.
Janssen W E et al Cytothepy, 2010; 12(3):418-24
Kassambara, A. (2011) Haemaologica, DOI: 10.3324/haematol.2011.046821.
Kingsmore S F. Nat Rev Drug Discov. 2006 April; 5(4):310-20
Kohlmann, A. et al., Journal of Clincal Oncology, 2010; 28(24):3858-65
Kosmider O et al., Haematogica, 2009; 94(12):1676-81
Le Carrour, T., (2010) The Open Bioinformatics Journal 4, 5-10.
Lee H S et al., Life Sciences, 2007; 80(7):690-8
Lee, H J et al., Gastroenterology, 2010; 139(1):213-25.
Livak K J & Schmittgen T D. Methods. 2001; 25(4):402-8
Mills, K. I., (2009) Blood, 114, 1063-1072.
Moreaux, J., (2011). Haematologica, 96, 574-582.
Orazi, A. & Germing, U. Leukemia, 2008; 22(7):1308-19
Piquemal et al, Genomics 2002; 80(3):361-71
Quéré R et al. Blood 2007; 109(10):4450-60
Reiter, A., (2009) Haematologica, 94, 1634-1638.
Rhodes, D. R., (2004) Neoplasia (New York, N.Y.), 6, 1-6.
Ricci, C. et al., Clincal Cancer Research, 2010; 16(8):2246-56
Rivals, E., (2007). Nucleic Acids Research, 35, e108-e108.
Schmitter, T., (2007) Infection and Immunity, 75, 4116-4126.
Such, E., et al. (2011). Haematologica, 96, 375-383.
Surasilp T et al., Mol Cell Probes. 2011
Tefferi A et al., Leukemia, 2009; 23(5):900-4
Theilgaard-Monch, K., (2011). Leukemia, 25, 909-920.
Toyokuni S et al., FEBS Letters 1995; 358(1):1-3
Vardiman, J. W., (2002). Blood, 100, 2292-2302
Woutets, B. J., (2009). Blood, 113, 291-298.

1. A method for in vitro determining the prognosis of chronic myelomonocytic leukaemia in a human patient suffering thereof, comprising the following steps:
a) measuring the expression level of at least two genes chosen in the group consisting of: G6PD (SEQ ID NO:2 or 3), 6PGD (SEQ ID NO:4), TKT (SEQ ID NO:9 or 10), CEACAM4 (SEQ ID NO: 11) and ELANE (SEQ ID NO:23) or homologous thereof, in a test sample of said human patient,
b) comparing said expression levels to the expression level of said at least two genes in a control sample of a known healthy human subject,
c) predicting the outcome of the chronic myelomonocytic leukaemia in said patient.

2. The method according to claim 1, wherein in step a) the expression level of the five genes G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) is measured.

3. The method according to claim 1, wherein higher expression level of at least the G6PD (SEQ ID NO:2 or 3), 6PGD (SEQ ID NO:4), TKT (SEQ ID NO:9 or 10), CEACAM4 (SEQ ID NO: 11) and ELANE (SEQ ID NO:23) genes in said test sample, as compared to said control sample, indicates a long-term survival of said human patient.

4. The method according to claim 1, wherein lower expression level of at least the G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) genes, indicates a short-term survival of said human patient.

5. The method according to claim 1, wherein said test and/or control sample is a sample of peripheral blood mononuclear cells (PBMC).

6. The method according to claim 1, wherein step a) comprises measuring the levels of the RNA transcripts or the cDNA of the said genes by employing nucleic acid based detection methods such as microarrays, quantitative PCR, DNA chips, hybridization wit labelled probes, or flow lateral dipstick.

7. The method according to claim 1, wherein step a) comprises measuring the levels of the respective proteins of the said genes by employing antibody-based detection methods such as immunohistochemistry or western blot analysis.

8. A kit for determining the prognosis of chronic myelomonocytic leukaemia in a human patient suffering thereof, comprising:
a) A reagent capable of specifically detecting the level of expression of at least two genes chosen among: G6PD (SEQ ID NO:2 or 3), 6PGD (SEQ ID NO:4), TKT (SEQ ID NO:9 or 10) CEACAM4 (SEQ ID NO:11), and ELANE (SEQ ID NO:23), and
b) Instructions for using said kit for determining the prognosis of chronic myelomonocytic leukaemia in said human patient.

9. A mRNA prognostic signature for predicting outcome of a patient suffering from chronic myelomonocytic leukaemia comprising one or more up-regulated mRNAs of the genes chosen in the group consisting of: G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) genes, as compared with mRNA of same genes expressed in normal cells.

10. A method for determining if patients suffering from chronic myelomonocytic leukaemia suffering will have a short-term survival or a long-term survival, comprising the steps of:
a) obtaining a test sample from said human patient,
b) determining the expression level of the at least two genes chosen in the group consisting of: the G6PD (SEQ ID NO:2 or 3), 6PGD (SEQ ID NO:4), TKT (SEQ ID NO:9 or 10), CEACAM4 (SEQ ID NO:11) and ELANE (SEQ ID NO:23) genes or homologous thereof in said test sample,
c) applying a predictive model for determining if said patient will have short-term survival or long-term survival.

11. Method according to claim 10, wherein the expression level of the five genes G6PD (SEQ ID NO:2 or 3), 6PGD (SEQ ID NO:4), TKT (SEQ ID NO:9 or 10), CEACAM4 (SEQ ID NO: 11) and ELANE (SEQ ID NO:23) is measured.

12. Method according to claim 10, wherein said predictive model comprises:
i) calculating the ratio between the expression level of the said genes in said test sample and the expression level of the same genes in a control sample of a known healthy human subject,
ii) comparing said ratio with cut-offs values for each gene and determining the dichotomisation factors for each gene,
iii) pondering said dichotomisation factors by predetermined beta-coefficient for each genes, and
iv) calculating an index I which is the sum of said dichotomised factors pondered by said beta-coefficients of said genes for said patient.

13. Method according to claim 12, wherein said cut-offs values are as follows:

| Gene name | Cut-offs |
|---|---|
| ELANE | 3.40 |
| G6PD | 1.15 |
| TKT | 1.2 |
| PGD | 1.22 |
| CEACAM4 | 1.34 |

and dichotomisation factors are calculated as follows:

| | Fold | |
|---|---|---|
| Gene name | change | Dichotomisation factor: D = |
| ELANE | b | +1 if b > 3.40; −1 if b ≦ 3.40 |
| G6PD | c | +1 if c > 1.15; −1 if c ≦ 1.15 |
| TKT | d | +1 if d > 1.2; −1 if d ≦ 1.2 |
| PGD | e | +1 if e > 1.22; −1 if e ≦ 1.22 |
| CEACAM4 | f | +1 if f > 1.34; −1 if f ≦ 1.34 |

and said beta-coefficients are as follows:

| Gene name | Beta-coefficient (β) |
|---|---|
| ELANE | 2.01784191521554 |
| G6PD | 1.28224877578792 |
| TKT | 1.35358578043486 |
| PGD | 1.71153730912409 |
| CEACAM4 | 2.0942792881 |

14. (canceled)

15. A nucleic acid microarray comprising nucleic acids specific for at least the 6 following genes: G6PD (SEQ ID NO:2 or 3), 6PGD (SEQ ID NO:4), TKT (SEQ ID NO:9 or 10) and CEACAM4 (SEQ ID NO: 11) and ELANE (SEQ ID NO:23) genes or homologous thereof.
